(19) United States
(12) Patent Application Publication
(10) Pub. No.: US 2025/0242001 A1
(43) Pub. Date: Jul. 31, 2025

(54) TISSUE KALLIKREIN-1 FOR TREATING PREGNANCY DISORDERS

(71) Applicant: DiaMedica Therapeutics, Inc., Minnetonka, MN (US)

(72) Inventors: David WAMBEKE, Edina, MN (US); Nick PAULSON, St. Paul, MN (US); Rick PAULS, Minnetonka, MN (US)

(21) Appl. No.: 19/041,724

(22) Filed: Jan. 30, 2025

Related U.S. Application Data
(60) Provisional application No. 63/626,954, filed on Jan. 30, 2024.

(51) Int. Cl. A61K 38/48; A61P 3/10; A61P 9/12; A61P 13/02; A61P 15/00

(52) U.S. Cl. CPC A61K 38/4853; A61P 3/10; A61P 9/12; A61P 13/02; A61P 15/00; C12Y 304/21035

(57) ABSTRACT

Provided are methods and compositions for using tissue kallikrein-1 (KLK1) to treat or prevent pregnancy disorders such as fetal growth restriction (FGR) and preeclampsia.

### CROSS-REFERENCE TO RELATED APPLICATIONS

This application claims the benefit under 35 U.S.C. § 119 (e) of U.S. Provisional Application No. 63/626,954, filed Jan. 30, 2024, which is incorporated by reference in its entirety.

### STATEMENT REGARDING THE SEQUENCE LISTING

The Sequence Listing XML associated with this application is provided in XML file format and is hereby incorporated by reference into the specification. The name of the XML file containing the Sequence Listing XML is DIAM_044_01US_ST26.xml. The XML file is about 5,479 bytes, was created on Jan. 28, 2025, and is being submitted electronically via USPTO Patent Center.

### BACKGROUND

### Technical Field

The present disclosure relates to methods and compositions for using tissue kallikrein-1 (KLK1) to treat or prevent pregnancy disorders such as fetal growth restriction (FGR) and preeclampsia.

### Description of the Related Art

Protein therapy with tissue kallikrein-1 (KLK1) has the potential to treat multiple diseases. For instance, KLK1 proteins such as the investigational drug DM199 are intended to restore normal levels of the naturally-occurring protein KLK1, which acts on interrelated mechanisms in the blood vessels and kidneys to improve circulation and overall function (see, for example, WO 2018/165551).

Complications of pregnancy, or pregnancy disorders, include a variety of underlying conditions and disorders related to hypertension, metabolic disorders, endothelial injury, reduced fetal blood flow, and other pathologies. Such complications can be life-threatening if untreated. However, many medications are considered unsafe during pregnancy because the medications cross the placenta and create potential safety issues for the fetus (see, for example, Tetro et al., Pharm Res. 35 (4): 71, 2018; Gedeon and Koren, Placenta. 27 (8): 861-8, 2006). For instance, front line anti-hypertensives such as angiotensin-converting enzyme (ACE) inhibitors and angiotensin II receptor blockers (ARBs) are contraindicated in pregnancy because these medications pass the placental barrier and harm the fetus (see, for example, Bullo et al., Hypertension. 60 (2): 444-450, 2012). Phosphodiesterase-5 (PDE-5) inhibitors such as sildenafil have been proposed for treating fetal growth restriction (FGS), but have also been shown to cross the placental barrier and associate with a statistically significant increase in the risk of neonatal pulmonary hypertension (see, for example, Pels et al., Eur J Pediatr. 181 (4): 1709-1718, 2022; Liu et al., J Pharm Pharm Sci. 27:13206, 2024). Also, most monoclonal antibody biologics readily cross the placenta, leading to concerns regarding their use during pregnancy and their impact on fetus and infant (Pham-Huy et al., CMAJ. 193 (29): E1129-1136, 2021).

Moreover, studies have shown that the passage of drugs across the placenta can no longer be predicted simply on the basis of their physical-chemical properties (see, for example, Staud et al., J Drug Target. 20 (9): 736-63, 2012), and guidance from sub-specialists on which drugs may be used and when they should be discontinued is often confusing at best (see, for example, Pham-Huy et al., supra). Thus, there is a strong need in the art to identify medications that are demonstrated to be reasonably safe to mother and fetus, do not pass the placental barrier, and which can address some of the underlying conditions and disorders associated with complications of pregnancy.

### BRIEF SUMMARY

Embodiments of the present disclosure relate to methods of treating or preventing a pregnancy disorder in a pregnant subject, comprising administering to the subject a pharmaceutical composition comprising one or more tissue kallikrein 1 (KLK1) polypeptides.

In some embodiments, the pregnancy disorder is fetal growth restriction (FGR) of the fetus in the pregnant subject. In some embodiments, the fetus has one or more of low body weight, low fundal height, low blood sugar levels, low body temperature, and/or elevated red blood cells. In some embodiments, the low body weight is an estimated fetal weight (EFW) of less than about the 10^{th} percentile for gestational age, for instance, as measured by prenatal ultrasound (ultrasonography-estimated EFW). In some embodiments, the FGR is severe FGR, for example, as characterized by an EFW of less than the 3^{rd} percentile. In some embodiments, the FGR is moderate FGR, optionally as characterized by an EFW of between the 3^{rd} and 9^{th} percentile.

In certain embodiments, the FGR is symmetrical. In some embodiments, the FGR is asymmetrical. In some embodiments, the fetus has one or more genetic abnormalities, optionally selected from aneuploidy, uniparental disomy, single-gene mutations, partial deletions or duplications, ring chromosome, and aberrant genomic imprinting. In some embodiments, the fetus has an infection, optionally selected from cytomegalovirus, toxoplasmosis, varicella-zoster virus, malaria, syphilis, and herpes simplex.

In particular embodiments, the pregnant subject has a condition or history selected from one or more of previous pregnancy with FGR, hypertension including previous pregnancy with preeclampsia, gestational or pregestational diabetes mellitus, systemic lupus erythematosus, antiphospholipid syndrome, cardiopulmonary or renal disease, anemia, malnourishment, sickle cell disease, a history of substance abuse (optionally alcohol, cocaine, nicotine, heroin, marijuana), a clinical history of anti-neoplastic drugs or radiation exposure, chronic antepartum hemorrhage, low pre-pregnancy weight or poor gestational weight gain, extremes of maternal age, short interpregnancy interval, high altitude residency, multiple gestations, uterine malformations, and assisted conception. In some embodiments, the placenta or umbilical cord in the subject is characterized with chromosomal placental mosaicism (CPM) (optionally presenting with placental trisomy, optionally trisomy 21), a placental anomaly (optionally bilobate or circumvallate placenta, small placenta, placental mesenchymal dysplasia), or an umbilical cord anomaly (optionally single artery, velamentous, marginal cord insertion). In certain embodiments, the FGR is early onset FGF (<about 32 weeks of pregnancy). In some embodiments, the FGR is late-onset FGR (>about 32 weeks of pregnancy).

In some embodiments, the pregnancy disorder is preeclampsia, eclampsia, or post-partum preeclampsia. In some embodiments, the subject has new-onset hypertension. In specific embodiments, the new-onset hypertension is characterized by:
a systolic blood pressure of 140 mm Hg or more or diastolic blood pressure of 90 mm Hg or more as measured on two or more occasions at least 4 to 6 hours apart; and/or
a systolic blood pressure of 160 mm Hg or more or diastolic blood pressure of 110 mm Hg or more as measured on two or more occasions less than 4 hours apart.

In some embodiments, the subject has proteinuria. In certain embodiments, the proteinuria is characterized by >about 0.3 grams or more of protein in a 24-hour urine sample, or a spot (random) urinary protein to creatinine ratio (UPCR) ≥about 0.3. In some embodiments, the subject has edema (optionally pulmonary edema or ankle edema, optionally pitting type). In some embodiments, the subject has a condition or history of kidney dysfunction (optionally oliguria, elevated creatinine levels), impaired liver function, thrombocytopenia (optionally a platelet count <about 100,000/μl), and/or cerebral or visual disturbances.

In some embodiments, the preeclampsia is early-onset (<about 32 weeks of pregnancy), optionally at or after about 20 weeks of pregnancy. In particular embodiments, the preeclampsia is late-onset (>about 32 weeks of pregnancy). In certain embodiments, the subject has non-severe preeclampsia. In some embodiments, the subject has severe preeclampsia. In specific embodiments, the severe preeclampsia is HELLP syndrome.

In particular embodiments, the subject with (or suspected of having) preeclampsia, eclampsia, or post-partum preeclampsia has increased serum levels of soluble fms-like tyrosine kinase 1 (sFlt-1), decreased serum levels of free placental growth factor (PlGF), increased ratio of sFlt-1/PlGf in serum, or any combination thereof. Specific methods comprise (a) determining serum levels of sFlt-1 and/or free PlGF in the subject; and (b) administering the pharmaceutical composition to the subject if the subject has increased serum levels of soluble fms-like tyrosine kinase 1 (sFlt-1), decreased serum levels of free placental growth factor (PlGF), increased ratio of sFlt-1/PlGf in serum, or any combination thereof.

In some embodiments, the pregnancy disorder is chronic hypertension, wherein the subject was hypertensive prior to pregnancy and was optionally undergoing hypertensive therapy that is discontinued during pregnancy. In particular embodiments, the hypertensive therapy is selected from angiotensin-converting-enzyme (ACE) inhibitor therapy, angiotensin II receptor blocker (ARB) therapy, and diuretic therapy optionally thiazide therapy.

In some embodiments, the pregnancy disorder is chronic hypertension superimposed with preeclampsia.

In some embodiments, the pregnancy disorder is gestational hypertension. In specific embodiments, the subject has a systolic blood pressure of 140 mm Hg or more and a diastolic blood pressure of 90 mm Hg or more that begins at or after about 20 weeks of pregnancy, and the subject does not have proteinuria.

In certain embodiments, the pregnancy disorder is post-partum hypertension, including wherein the subject has a systolic blood pressure of 140 mm Hg or more and a diastolic blood pressure of 90 mm Hg or more that begins or is present after childbirth, for example, which begins or is present within about 48 hours after childbirth, or within about 1, 2, 3, 4, 5, or 6 weeks after childbirth.

In some embodiments, the subject has hypertension or a history of hypertension, and wherein the method comprises administering the pharmaceutical composition as blood pressure control for a planned delivery about 72 hours before the delivery.

In particular embodiments, the pregnancy disorder is pregnancy-associated stroke, including ischemic stroke or hemorrhagic stroke. In specific embodiments, the pregnancy-associated stroke is post-partum stroke, including wherein the stroke occurs within about 48 hours of childbirth, within about 1, 2, 3, 4, 5, 6, 7, 8, 9, or 10 days after childbirth, or within about 1, 2, 3, 4, 5, or 6 weeks after childbirth.

In some embodiments, the subject has low serum KLK1 levels, optionally below the limit of detection, optionally below about 1 ng/ml.

In specific embodiments, the pharmaceutical composition comprises DM199.

In some embodiments, the pharmaceutical composition comprises a first KLK1 polypeptide and a second KLK1 polypeptide, wherein the first KLK1 polypeptide has three glycans attached at three different positions per polypeptide and the second KLK1 polypeptide has two glycans attached at two different positions per polypeptide, and wherein the first KLK1 polypeptide and the second KLK1 polypeptides are present in the pharmaceutical composition at a ratio of about 45:55 to about 55:45. In particular embodiments, one or more of the glycans are N-linked glycans. In some embodiments, one or more of the glycans are attached at amino acid residues 78, 84, or 141 of KLK1 as defined by SEQ ID NO: 3 or 4. In certain embodiments, the three glycans of the first KLK1 polypeptide are N-linked glycans at residues 78, 84, and 141. In some embodiments, the two glycans of the second KLK1 polypeptide are N-linked glycans at residues 78 and 84 but not 141. In some embodiments, the first KLK1 polypeptide and the second KLK1 polypeptides are present in the pharmaceutical composition at a ratio of about 50:50.

In certain embodiments, the one or more KLK1 polypeptide(s) are recombinant KLK polypeptides, mature KLK1 polypeptides, human KLK1 (hKLK1) polypeptides, or any combination thereof.

In some embodiments, the hKLK1 polypeptide(s) comprise, consist, or consist essentially of amino acid residues 78-141 of SEQ ID NO:1 or amino acids residues 78-141 SEQ ID NO: 2, or an active fragment thereof, or an active variant having at least about 90, 95, 96, 97, 98, or 99% sequence identity to amino acid residues 78-141 of SEQ ID NO:1 or amino acids residues 78-141 SEQ ID NO:2. In certain embodiments, the hKLK1 polypeptide(s) comprise, consist, or consist essentially of amino acid residues 25-262 of SEQ ID NO:1 or amino acid residues 25-262 of SEQ ID NO:2, or an active fragment thereof, or an active variant having at least about 90, 95, 96, 97, 98, or 99% sequence identity to amino acid residues 25-262 of SEQ ID NO: 1 or amino acid residues 25-262 of SEQ ID NO:2. In particular embodiments, the KLK1 polypeptide(s) comprise an amino acid sequence having at least about 90, 95, 96, 97, 98, or 99% sequence identity to amino acid residues 25-262 of SEQ ID NO:2, and wherein the KLK1 polypeptide(s) comprises E145 and/or A188. In specific embodiments, the KLK1 polypeptide(s) comprise an amino acid sequence having at least about 90, 95, 96, 97, 98, or 99% sequence identity to amino acid residues 25-262 of SEQ ID NO:2, and wherein the KLK1 polypeptide(s) comprises Q145 and/or V188.

In some embodiments, the pharmaceutical composition is formulated or administered at a total KLK1 polypeptide dosage of about 0.25 or 0.5 μg/kg to about 10.0 μg/kg or to about 15 μg/kg or to about 20 μg/kg, optionally about 0.1, 0.2, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19 or 20 μg/kg. In certain embodiments, the pharmaceutical composition is formulated or administered at a total KLK1 polypeptide intravenous dosage of about 0.1 or 0.25 or 0.5 g/kg. In some embodiments, the pharmaceutical composition is formulated or administered at a total KLK1 polypeptide subcutaneous dosage of about 3.0 μg/kg or up to about 15 μg/kg. In some embodiments, the pharmaceutical composition is administered as a dosing regimen of about a total KLK1 polypeptide intravenous dosage of about 0.1 or 0.25 or 0.5 μg/kg per day (for example, as an approximately 30 minute IV infusion), and a total KLK1 polypeptide subcutaneous dosage of about 3.0 μg/kg or up to about 15 μg/kg every 2, 3, or 4 days, optionally every 3 days.

Certain embodiments include subcutaneously or intravenously administering the pharmaceutical composition to the subject. In some embodiments, the pharmaceutical composition comprises a pharmaceutically acceptable excipient, diluent, adjuvant, or carrier. In some embodiments, the pharmaceutical composition is substantially free of other glycosylated isoforms (glycoforms) of KLK1. In certain embodiments, the pharmaceutical composition has endotoxin levels of less than about 1 EU/mg protein, host cell protein of less than about 100 ng/mg total protein, host cell DNA of less than about 10 pg/mg total protein, and/or is substantially free of aggregates (greater than about 95% appearing as a single peak by SEC HPLC).

In some embodiments, administering the pharmaceutical composition improves one or more clinical parameters in the subject. In particular embodiments, the pregnancy disorder is FGR, and wherein the one or more clinical parameters are selected from serum KLK1 levels of about 1-5 ng/ml in the pregnant subject and increased fetal body weight for gestational age (optionally increased EFW percentile of about or greater than 10^{th}, 15^{th}, 20^{th}, 25^{th}, 30^{th}, 35^{th}, 40^{th}, 50^{th}, or greater percentile for gestational age). In certain embodiments, the pregnancy disorder is preeclampsia, and wherein the one or more clinical parameters are selected from serum KLK1 levels of about 1-5 ng/ml, decreased systolic and/or diastolic blood pressure, decreased proteinuria, and/or decreased edema in the pregnant subject.

### BRIEF DESCRIPTION OF THE FIGURES

FIG. 1 summarizes DM199-mediated reductions in systolic blood pressure in cohorts of hypertensive patients at day 95 of treatment.

FIG. 2 shows that DM199 reduces blood glucose levels in human patients.

FIG. 3 shows the results of a placental transfer study of DM199 in rats.

FIG. 4 shows that DM199 infusion reduces the average systolic blood pressure in five (5) patients with preeclampsia (Phase 2).

FIG. 5 shows that DM199 infusion reduces the uterine artery pulsality index in all 3 (three) preeclampsia patients for which this data was available, a result that evidences improved blood flow to the fetus.

### DETAILED DESCRIPTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, chemical synthesis, and tissue culture, among others. Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

The term “composition” includes “pharmaceutical compositions”, “therapeutic compositions”, “solutions” such as IV solutions, “formulations”, and “dosage forms”.

The terms “endotoxin free” or “substantially endotoxin free” relate generally to dosage forms, compositions, solvents, devices, and/or vessels that contain at most trace amounts (e.g., amounts having no clinically adverse physiological effects to a subject) of endotoxin, and preferably undetectable amounts of endotoxin. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art. Also included are methods of producing KLK1 polypeptides in and isolating them from eukaryotic cells such as mammalian cells to reduce, if not eliminate, the risk of endotoxins being present in a composition of the invention. Preferred are methods of producing KLK1 polypeptides in and isolating them from recombinant cells grown in chemically defined, serum free media.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/ml, or EU/mg protein. Typically, 1 ng lipopolysaccharide (LPS) corresponds to about 1-10 EU.

The “half-life” of an agent such as a KLK1 polypeptide (e.g., DM199) can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the levels of agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulate” and “alter” include “increase,” “enhance” or “stimulate,” as well as “decrease”, “inhibit”, or “reduce,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7, 1.8, etc.) the amount or level produced by a control composition, sample or test subject. A “decreased”, “inhibited, or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount or level produced a control composition, sample or test subject. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

A result is typically referred to as “statistically significant” if it is unlikely to have occurred by chance. The significance level of a test or result relates traditionally to the amount of evidence required to accept that an event is unlikely to have arisen by chance. In certain cases, statistical significance may be defined as the probability of making a decision to reject the null hypothesis when the null hypothesis is actually true (a decision known as a Type I error, or “false positive determination”). This decision is often made using the p-value: if the p-value is less than the significance level, then the null hypothesis is rejected. The smaller the p-value, the more significant the result. Bayes factors may also be utilized to determine statistical significance (see Goodman, Ann Intern Med. 130:1005-13, 1999).

The term “solubility” refers to the property of a KLK1 polypeptide provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 6.0, pH 7.0, pH 7.4, pH 8.0 or pH 9.0. In certain embodiments, solubility is measured in water or a physiological buffer such as PBS or NaCl (with or without NaP). In specific embodiments, solubility is measured at relatively lower pH (for example, pH 6.0) and relatively higher salt (for example, 500 mM NaCl and 10 mM NaP). In certain embodiments, solubility is measured in a biological fluid (solvent) such as blood or serum. In certain embodiments, the temperature can be about room temperature (for example, about 20, about 21, about 22, about 23, about 24, or about 25° C.) or about body temperature (37° C.). In certain embodiments, a KLK1 polypeptide has a solubility of at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, or at least about 60 mg/ml at room temperature or at 37° C.

“Substantially” or “essentially” means nearly totally or completely, for instance, 95%, 96%, 97%, 98%, 99% or greater of some given quantity.

“Treatment” or “treating,” as used herein, includes any desirable effect on the symptoms or pathology of a disease or disorder, and may include even minimal changes or improvements in one or more measurable markers of the disease or disorder being treated. Also included are “prophylactic” or “preventative” treatments, which can be directed at reducing the rate of progression towards the disease or disorder, delaying the onset of that disease or disorder, or reducing the severity of its onset. “Treating” or “preventing” or “prophylaxis” does not necessarily indicate complete eradication, cure, or prevention of the disease or disorder, or associated symptoms thereof. The subject receiving this treatment is any subject in need thereof. Exemplary markers of clinical improvement will be apparent to persons skilled in the art.

As used herein, the terms “therapeutically effective amount”, “therapeutic dose,” “prophylactically effective amount,” or “diagnostically effective amount” is the amount of an agent such as a KLK1 polypeptide (e.g., DM199) needed to elicit the desired biological response following administration.

A “subject,” as used herein, includes any animal that exhibits a symptom, or is at risk for exhibiting a symptom, which can be treated with a KLK1 polypeptide or a dosage form thereof. Suitable subjects (patients) include laboratory animals (such as mouse, rat, rabbit, or guinea pig), farm animals, and domestic animals or pets (such as a cat or dog). Non-human primates and, preferably, human patients, are included.

By “isolated” is meant material that is substantially or essentially free from components that normally accompany it in its native state. For example, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances such as host cell proteins or nucleic acids.

A “wild type” or “reference” sequence or the sequence of a “wild type” or “reference” protein/polypeptide may be the reference sequence from which variant polypeptides are derived through the introduction of changes. In general, the “wild type” amino acid sequence for a given protein is the sequence that is most common in nature. Similarly, a “wild type” gene sequence is the polynucleotide sequence for that gene which is most commonly found in nature. Mutations can be introduced into a “wild type” gene (and thus the protein it encodes) either through natural processes or through human induced means.

Each embodiment in this specification is to be applied to every other embodiment unless expressly stated otherwise.

Complications of Pregnancy (Pregnancy Disorders). Embodiments of the present disclosure relate to methods of treating or preventing a pregnancy disorder in a pregnant subject, comprising administering to the subject a pharmaceutical composition comprising one or more tissue kallikrein 1 (KLK1) polypeptides. In some embodiments, the subject has low serum KLK1 levels, for example, below the practical limit of detection. In some instances, the subject has serum KLK1 levels below about 1 ng/ml.

In certain embodiments, the pregnancy disorder is fetal growth restriction (FGR), also referred to as intrauterine growth restriction (IUGR) (see, for example, Chew and Verma, Treasure Island (FL): StatPearls Publishing; 2023; Romo et al., J. Intrauterine growth retardation (IUGR): epidemiology and etiology. Pediatr Endocrinol Rev. 2009 February; 6 Suppl 3:332-6; Sharma et al., Intrauterine Growth Restriction: Antenatal and Postnatal Aspects. Clin Med Insights Pediatr. 2016; 10:67-83; Faraci et al., J Prenat Med. 2011 April; 5 (2): 31-3). In certain embodiments, the fetus has one or more of low body weight, low fundal height, low blood sugar levels, low body temperature, and/or elevated red blood cells. “Fundal height” refers to the distance between the pubic symphysis and the top of the uterus, and can be used to estimate gestational age (see, for example, Morse et al., Best Pract Res Clin Obstet Gynaecol. 2009 December; 23 (6): 809-18). Typically, the feature of a low body weight refers to an estimated fetal weight (EFW) of less than about the 10^{th} percentile for gestational age (see, for example, Sharma et al., 2016, supra). In certain embodiments, EFW is measured by prenatal ultrasound, for instance, to generate an ultrasonography-estimated EFW.

The severity of FGR can be determined according to the EFW percentile (see, for example, Lee et al., Pediatrics. 2003 June; 111 (6 Pt 1): 1253-61). In some embodiments, the FGR is “severe FGR”, which is typically characterized by an EFW of less than the 3^{rd} percentile. In certain embodiments, the FGR is “moderate FGR”, which is typically characterized by an EFW of between the 3^{rd} and 9^{th} percentile.

In some embodiments, the FGR is symmetrical. In symmetrical FGR, all growth parameters are proportionally reduced. Symmetrical FGR constitutes about 20% to 30% of all FGR cases. Poor placental function is a well-established cause, as are adverse intrauterine conditions beginning in the early pregnancy (first trimester) that may cause fetal nutrient restriction. Examples include smoking, cocaine use, chronic hypertension, anemia, and chronic diabetes mellitus. Chromosome anomalies, such as aneuploidy, are also a major cause of symmetrical FGR (see Faraci et la., 2011, supra). TORCH infection (Toxoplasma gondii, cytomegalovirus, herpes simplex virus, varicella-zoster virus, Treponema, and others) contracted prenatally can be present in about 5% to 15% of cases with symmetrical FGR (see, for example, Longo et al., Early Hum Dev. 2014 March; 90 Suppl 1: S42-4).

In some embodiments, the FGR is asymmetrical. In asymmetrical FGR, the abdominal circumference is reduced below the 10^{th} percentile, while other measurements are relatively preserved and may be within normal limits. Asymmetrical FGR about 70% to 80% of all FGR cases, and the timing of intrauterine insult is typically at the late second or third trimester of pregnancy. The growth restriction is disproportionate, with relative preservation of head circumference (fetal brain) and reduced abdominal circumference (fetal liver), resulting in an increased brain to the liver ratio (BLR). Preeclampsia is a well-recognized cause of asymmetrical FGR, which generally develops after 20 weeks of gestation and is characterized by hypertension and sometimes proteinuria (see, for example, Uzan et al., Vasc Health Risk Manag. 2011; 7:467-74). Chronic hypertension leads to placental vascular remodeling, vascular sclerosis, and ischemia, impeding blood flow to the fetus. As a result, the fetal liver glycogen and body adipose tissues diminish while the brain continues to grow normally with a preferential blood supply.

In some instances, the FGR is associated with fetal causes, maternal causes, and/or placental/umbilical cord causes. Examples of fetal causes include situations in which the fetus has one or more genetic abnormalities such as aneuploidy, uniparental disomy, single-gene mutations, partial deletions or duplications, ring chromosome, and/or aberrant genomic imprinting. Also included are situations in which the fetus has an infection, such as cytomegalovirus, toxoplasmosis, varicella-zoster virus, malaria, syphilis, and/or herpes simplex.

Examples of maternal causes include situations in which the pregnant subject has a condition or history such as previous pregnancy with FGR, hypertension including previous pregnancy with preeclampsia, gestational or pregestational diabetes mellitus, systemic lupus erythematosus, antiphospholipid syndrome, cardiopulmonary or renal disease, anemia, malnourishment, sickle cell disease, a history of substance abuse (for example, alcohol, cocaine, nicotine, heroin, marijuana), a clinical history of anti-neoplastic drugs or radiation exposure, chronic antepartum hemorrhage, low pre-pregnancy weight or poor gestational weight gain, extremes of maternal age, short interpregnancy interval, high altitude residency, multiple gestations, uterine malformations, and/or assisted conception. Also, mothers who were themselves growth restricted carry a significantly higher risk of carrying or delivering FGR neonates.

Examples of placental/umbilical cord causes include situations in which the placenta or umbilical cord in the subject is characterized with chromosomal placental mosaicism (CPM) (for example, presenting with placental trisomy, optionally trisomy 21), a placental anomaly (for example, bilobate or circumvallate placenta, small placenta, placental mesenchymal dysplasia), and/or an umbilical cord anomaly (for example, single artery, velamentous, marginal cord insertion).

In some embodiments, the FGR is “early onset FGF”, which is characterized as being <about 32 weeks of pregnancy. In certain embodiments, the FGR is “late-onset FGR”, which is characterized as being >about 32 weeks of pregnancy.

In some embodiments, the pregnancy disorder is preeclampsia, eclampsia, or post-partum preeclampsia (see, for example, Karrar and Hong, Treasure Island (FL): StatPearls Publishing; 2023; Gestational Hypertension and Preeclampsia: ACOG Practice Bulletin, Number 222. Obstet Gynecol. 2020 June; 135 (6): e237-e260; Homer et al., J Hypertens. 2008 February; 26 (2): 295-302; Phipps et al., Nat Rev Nephrol. 2019 May; 15 (5): 275-289; Amaral et al., Curr Hypertens Rep. 2017 August; 19 (8): 61). “Preeclampsia” refers generally to a hypertensive disease that occurs during pregnancy, typically in a pregnant and hypertensive subject with previously-normal blood pressure—that is, a pregnant subject with “new-onset hypertension”. “Eclampsia” refers to the onset of seizures (convulsions) in a subject with preeclampsia. “Post-partum” preeclampsia refers to “preeclampsia” that develops after childbirth, for example, within about 48 hours after childbirth, or sometimes up to about or within about 1, 2, 3, 4, 5, or 6 weeks after childbirth. Thus, in certain embodiments, the subject has preeclampsia, eclampsia (seizures), or post-partum preeclampsia.

In certain embodiments, a pregnant subject with preeclampsia, eclampsia, or post-partum preeclampsia has new-onset hypertension, which is typically characterized by: a systolic blood pressure of 140 mm Hg or more or diastolic blood pressure of 90 mm Hg or more as measured on two or more occasions at least 4 to 6 hours apart; and/or a systolic blood pressure of 160 mm Hg or more or diastolic blood pressure of 110 mm Hg or more as measured on two or more occasions less than 4 hours apart.

In some instances, a subject with preeclampsia, eclampsia, or post-partum preeclampsia has proteinuria, which refers to excess proteins in urine. In some instances, the proteinuria is characterized by >about 0.3 grams or more of protein in a 24-hour urine sample, or a spot (random) urinary protein to creatinine ratio (UPCR) ≥about 0.3. However, not all instances of preeclampsia are associated with proteinuria. In such cases, where new-onset hypertension is observed in the absence of proteinuria, other new-onset symptoms such as thrombocytopenia, renal insufficiency, pulmonary edema, impaired liver function, or new-onset headache with or without visual disturbance can be used for diagnosis

In some instances, a subject with preeclampsia, eclampsia, or post-partum preeclampsia has edema or swelling. Examples of edema include pulmonary edema and ankle edema (for example, pitting type). In some instances, the subject has a condition or history of kidney dysfunction (optionally oliguria, elevated creatinine levels), impaired liver function, thrombocytopenia (optionally a platelet count <about 100,000/μl), and/or cerebral or visual disturbances.

In some embodiments, the preeclampsia is early-onset (<about 32 weeks of pregnancy), for example, starting at or after about 20 weeks of pregnancy. In certain embodiments, the preeclampsia is late-onset (>about 32 weeks of pregnancy).

In some embodiments, the subject has non-severe preeclampsia. In certain embodiments, the subject has severe preeclampsia. For example, in some embodiments, the subject has a severe form of preeclampsia often referred to as HELLP (hemolysis, elevated liver enzymes, and low platelet count) syndrome (see, for example, Adorno et al., Crit Care Nurs Clin North Am. 34 (3): 277-288, 2022).

In certain embodiments, the subject with preeclampsia has increased serum levels of soluble fms-like tyrosine kinase 1 (sFlt-1), decreased serum levels of free placental growth factor (PlGF), increased ratio of sFlt-1/PlGf in serum, or any combination thereof (see, for example, Velegrakis et al., Int J Mol Med. 52 (4): 89), 2023). In silico analysis (EXPASY-PEPTIDE CUTTER, data not shown) suggests that KLK1 should cleave sFlt-1 at numerous sites (˜37 Arg cleavage sites in the full Flt-1 sequence), further evidencing its therapeutic utility in preeclampsia where increased levels of sFlt-1 are associated with disease.

In some embodiments, the pregnancy disorder is chronic hypertension, including wherein the subject is/was hypertensive prior to pregnancy and was ideally undergoing hypertensive therapy that is being discontinued during pregnancy. Examples of hypertensive therapies include angiotensin-converting-enzyme (ACE) inhibitor therapies, angiotensin II receptor blocker (ARB) therapies, and diuretic therapies, for example, thiazide therapies.

In some embodiments, the pregnancy disorder is chronic hypertension superimposed with preeclampsia (see, for example, Kametas et al., Am J Obstet Gynecol. 226 (2S): S1182-S1195, 2022).

In particular embodiments, the pregnancy disorder is gestational hypertension. Gestational hypertension is often indicated in a subject that has a systolic blood pressure of 140 mm Hg or more and a diastolic blood pressure of 90 mm Hg or more which begins at or after about 20 weeks of pregnancy. A subject with gestational hypertension typically does not have proteinuria.

In certain embodiments, the pregnancy disorder is post-partum hypertension, including wherein the subject has a systolic blood pressure of 140 mm Hg or more and a diastolic blood pressure of 90 mm Hg or more that begins or is present after childbirth. In some embodiments, “post-partum” hypertension in a subject begins or is present within about 48 hours after childbirth, or within about 1, 2, 3, 4, 5, or 6 weeks after childbirth.

Particular embodiments include methods of reducing (e.g., the risk of developing) post-partum hypertension as measured at about one year after childbirth, for example, by administering KLK1 (e.g., DM199) for about or up to about 6, 7, 8, 9, 10, 11, or 12 weeks after childbirth.

In particular embodiments, the pregnancy disorder is pregnancy-associated stroke, or maternal stroke associated with pregnancy (see, for example, Karjalainen et al., BMC Pregnancy Childbirth. 19:187, 2019; and Miller, Continuum (Minneap Minn). 28:93-121, 2022). Examples of pregnancy-associated stroke include ischemic stroke and hemorrhagic stroke. In specific embodiments, the pregnancy-associated stroke is “post-partum” stroke, including wherein the stroke occurs within about 48 hours of childbirth, within about 1, 2, 3, 4, 5, 6, 7, 8, 9, or 10 days after childbirth, or within about 1, 2, 3, 4, 5, or 6 weeks after childbirth.

Certain embodiments include administering a pharmaceutical composition described herein as blood pressure control for a planned delivery. Thus, in certain embodiments, the subject has hypertension or a history of hypertension, and the method comprises administering the pharmaceutical composition as blood pressure control for a planned delivery about or within about 72 hours before the planned delivery.

The KLK1 polypeptides and related pharmaceutical compositions can be used to treat or prevent any one or combination of the foregoing complications of pregnancy, or pregnancy disorders, as described herein.

Tissue Kallikrein-1 (KLK1) Polypeptides. The compositions described herein comprise one or more tissue kallikrein-1 or KLK1 polypeptides. Tissue kallikreins are members of a gene super family of serine proteases comprising at least 15 separate and distinct proteins (named tissue kallikrein 1 through 15) (Yousef et al., 2001, Endocrine Rev; 22:184-204). Tissue kallikrein-1 is a trypsin-like serine protease. In humans and animal tissues, tissue kallikrein-1 cleaves kininogen into lysyl-bradykinin (also known as kallidin), a decapeptide kinin having physiologic effects similar to those of bradykinin. Bradykinin is a peptide that causes blood vessels to dilate and therefore causes blood pressure to lower. Kallidin is identical to bradykinin with an additional lysine residue added at the N-terminal end and signals through the bradykinin receptor.

The KLK1 gene encodes a single pre-pro-enzyme that is 262 amino acid residues in length and that includes the “pre-” sequence (residues 1-18) and the “pro-” sequence (residues 19-24), which is activated by trypsin-like enzymes. The “mature” and “active” form human KLK1 is a glycoprotein of about 238 amino acid residues (residues 25-262) with a molecular weight of 26 kDa and a theoretical μl of 4.6. KLK1 has five disulfide bonds in its tertiary structure that are believed to be responsible for the protein's high stability, both against trypsin digestion and heat inactivation.

The amino acid sequence of full-length tissue kallikrein-1 is available for a wide variety of species, including, but not limited to, human (SEQ ID NO:1 and SEQ ID NO:2), mouse (see, for example, GenBank: AAA39349.1, Feb. 1, 1994); domestic cat (see, for example, NCBI Reference Sequence: XP_003997527.1, Nov. 6, 2012); gorilla (see, for example, NCBI Reference Sequence: XP_004061305.1, Dec. 3, 2012); cattle (see, for example, GenBank: AAI51559.1, Aug. 2, 2007); dog (see, for example, CBI Reference Sequence: NP_001003262.1, Feb. 22, 2013); rat (see, for example, GenBank: CAE51906.1, Apr. 25, 2006); and olive baboon (see, for example, NCBI Reference Sequence: XP_003916022.1, Sep. 4, 2012). KLK1 is functionally conserved across species in its capacity to release the vasoactive peptide, Lys-bradykinin, from low molecular weight kininogen. A tissue kallikrein-1 polypeptide of the present invention may have any of the known amino acid sequences for KLK1, or a fragment or variant thereof.

In certain embodiments, the KLK1 polypeptide is a “mature” KLK1 polypeptide. In certain embodiments, the KLK1 polypeptide is a human KLK1 polypeptide, optionally a mature human KLK1 polypeptide. In particular embodiments, the KLK1 polypeptide is a recombinant human polypeptide, for example, a recombinant human KLK1 polypeptide, optionally in the mature form. Recombinant human KLK1 (rhKLK1) can provide certain advantages over other sources of KLK1, such as urinary KLK1 (e.g., human KLK1 isolated from human urine), including a homogenous preparation of rhKLK1, simpler regulatory path to licensure, and options to alter the amino acid sequence or glycosylation pattern based on cell culture conditions.

Exemplary amino acid sequences of human tissue kallikrein-1 (hKLK1) polypeptides are provided in Table K1 below.

TABLE K1
Exemplary KLK1 Sequences

| | | SEQ ID |
|---|---|---|
| Source | Sequence | NO: |
| Human | MWFLVLCLALSLGGTGAAPPIQSRIVGGWEC | 1 |
| KLK1 | EQHSQPWQAALYHFSTFQCGGILVHRQWVLT | |
| | AAHCISDNYQLWLGRHNLFDDENTAQFVHVS | |
| | ESFPHPGFNMSLLENHTRQADEDYSHDLMLL | |
| | RLTEPADTITDAVKVVELPTEEPEVGSTCLA | |
| | SGWGSIEPENFSFPDDLQCVDLKILPNDECK | |
| | KAHVQKVTDFMLCVGHLEGGKDTCVGDSGGP | |
| | LMCDGVLQGVTSWGYVPCGTPNKPSVAVRVL | |
| | SYVKWIEDTIAENS | |
| Human | MWFLVLCLALSLGGTGAAPPIQSRIVGGWEC | 2 |
| KLK1 | EQHSQPWQAALYHFSTFQCGGILVHRQWVLT | |
| variant | AAHCISDNYQLWLGRHNLFDDENTAQFVHVS | |
| | ESFPHPGFNMSLLENHTRQADEDYSHDLMLL | |
| | RLTEPADTITDAVKVVELPTQEPEVGSTCLA | |
| | SGWGSIEPENFSFPDDLQCVDLKILPNDECK | |
| | KVHVQKVTDFMLCVGHLEGGKDTCVGDSGGP | |
| | LMCDGVLQGVTSWGYVPCGTPNKPSVAVRVL | |
| | SYVKWIEDTIAENS | |
| Human | ivggweceqhsqpwqaalyhfstfqcggilv | 3 |
| mature | hrqwvltaahcisdnyqlwlgrhnlfddent | |
| KLK1 | aqfvhvsesfphpgfnmsllenhtrqadedy | |
| | shdlmllrltepadtitdavkvvelpteepe | |
| | vgstclasgwgsiepenfsfpddlqcvdlki | |
| | lpndeckkahvqkvtdfmlcvghleggkdtc | |
| | vgdsggplmcdgvlqgvtswgyvpcgtpnkp | |
| | svavrvlsyvkwiedtiaens | |
| Human | IVGGWECEQHSQPWQAALYHFSTFQCGGILV | 4 |
| mature | HRQWVLTAAHCISDNYQLWLGRHNLFDDENT | |
| KLK1 | AQFVHVSESFPHPGFNMSLLENHTRQADEDY | |
| variant | SHDLMLLRLTEPADTITDAVKVVELPTQEPE | |
| | VGSTCLASGWGSIEPENFSFPDDLQCVDLKI | |
| | LPNDECKKVHVQKVTDFMLCVGHLEGGKDTC | |
| | VGDSGGPLMCDGVLQGVTSWGYVPCGTPNKP | |
| | SVAVRVLSYVKWIEDTIAENS | |

In certain embodiments, a KLK1 polypeptide comprises, consists, or consists essentially of SEQ ID NO: 1-3 or 4, or residues 1-262, residues 19-262, or residues 25-262 of SEQ ID NO: 1 or SEQ ID NO:2, including fragments and variants thereof. Amino acids 1 to 18 of SEQ ID NO:1 and 2 represent the signal peptide, amino acids 19 to 24 represent propeptide sequences, and amino acids 25 to 262 represent the mature peptide. Thus, the preproprotein includes a presumptive 17-amino acid signal peptide, a 7-amino acid proenzyme fragment and a 238-amino acid mature KLK1 protein.

A comparison between SEQ ID NO:1 and SEQ ID NO:2 (or SEQ ID NO:3 and SEQ ID NO: 4) shows two amino acid differences between the two hKLK1 amino acid sequences. Single-nucleotide polymorphism (SNPs) between the two individuals within a species account for an E to Q substitution at amino acid residue 145 of 262 and an A to V substitution at position 188 of 262. SEQ ID NO:1 has an E (glutamic acid) at position 145 and an A (alanine) at position 188, while SEQ ID NO:2 has a Q (glutamine) at position 145 and a V (valine) at position 188. In some embodiments, KLK1 polypeptide has an E at position 145; a Q at position 145; an A at position 188; an A at position 188; an E at position 145 and an A at position 188; a Q at position 145 and a V at position 188; a Q at position 145 and an A at position 188; or an E at position 145 and a V at position 188.

As noted above, certain embodiments include active variants and fragments of reference KLK1 polypeptide. A “variant” of a starting or reference polypeptide is a polypeptide that has an amino acid sequence different from that of the starting or reference polypeptide. Such variants include, for example, deletions from, insertions into, and/or substitutions of residues within the amino acid sequence of the polypeptide of interest. A variant amino acid, in this context, refers to an amino acid different from the amino acid at the corresponding position in a starting or reference polypeptide sequence. Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

In some embodiments, a KLK polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to a reference sequence, such as, for example, an amino acid sequence described herein (for example, SEQ ID NOs: 1-4).

In some aspects, a KLK1 polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to SEQ ID NO:1 or 3, or to a fragment of SEQ ID NO: 1 or 3, such as for example, residues 25-262 or residues 78-141 of SEQ ID NO: 1. Such a KLK1 polypeptide may have an E or a Q at amino acid residue 145, and/or an A or a V at position 188.

In some aspects, a KLK1 polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to SEQ ID NO:2 or 4, or to a fragment of SEQ ID NO:2 or 4, such as for example, residues 25-262 or residues 78-141 of SEQ ID NO:2. Such a KLK1 polypeptide may have an E or a Q at amino acid residue 145, and/or an A or a V at position 188.

“Percent (%) amino acid sequence identity” with respect to a polypeptide is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, California.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Variants may also include heterologous sequences or chemical modifications which are added to the reference KLK1 polypeptide, for example, to facilitate purification, improve metabolic half-life, or make the polypeptide easier to identify. Examples include affinity tags such as a His-tag, Fc regions, and/or a PEGylation sequence and PEG.

The term “fragment” includes smaller portions of a KLK1 polypeptide (or variants thereof) that retain the activity of a KLK1 polypeptide. Fragments includes, for example, a KLK1 polypeptide fragment that ranges in size from about 20 to about 50, about 20 to about 100, about 20 to about 150, about 20 to about 200, or about 20 to about 250 amino acids in length. In other embodiments, a KLK1 polypeptide fragment ranges in size from about 50 to about 100, about 50 to about 150, about 50 to about 200, or about 50 to about 250 amino acids in length. In other embodiments, a KLK1 polypeptide fragment ranges in size from about 100 to about 150, about 100 to about 200, about 100 to about 250, about 150 to about 175, about 150 to about 200, or about 150 to about 250 amino acids in length. In other illustrative embodiments, a KLK1 polypeptide fragment ranges in size from about 200 to about 250 amino acids in length. Certain embodiments comprise a polypeptide fragment of a full-length KLK1 of about, up to about, or at least about 50, 60, 70, 80, 90, 100, 110, 120, 130, 140, 150, 160, 170, 180, 190, 200, 210, 220, 230, 240, 250 or more (e.g., contiguous) amino acid residues. In some embodiments, a fragment may have residues 25-262 or residues 78-141 of a preproprotein sequence. In some embodiments, a fragment may be any such fragment size, as described above, of SEQ D NO: 1 or SEQ ID NO:2.

In some instances, fragments and variants of a KLK1 polypeptide retain the enzymatic capacity to release the vasoactive peptide, Lys-bradykinin, from low molecular weight kininogen. In some embodiments, an active variant or fragment retains serine protease activity of a KLK1 polypeptide that releases kallidin from a higher molecular weight precursor such as kininogen, or that cleaves a substrate similar to kininogen such as D-val-leu-arg-7 amido-4-trifluoromethylcoumarin to release a colorimetric or fluorometric fragment. The protease activity of KLK1 polypeptides can be measured in an enzyme activity assay by measuring either the cleavage of low-molecular-weight kininogen, or the generation of lys-bradykinin. In one assay format, a labeled substrate is reacted with the KLK1 glycoform, and the release of a labeled fragment is detected. One example of such a fluorogenic substrate suitable for KLK1 measurement of activity is D-val-leu-arg-7 amido-4-trifluoromethylcoumarin (D-VLR-AFC, FW 597.6) (Sigma, Cat #V2888 or Ana Spec Inc Cat #24137). When D-VLR-AFC is hydrolyzed, the free AFC produced in the reaction can be quantified by fluorometric detection (excitation 400 nm, emission 505 nm) or by spectrophotometric detection at 380 nm (extinction coefficient=12,600 at pH 7.2). Other methods and substrates may also be used to measure KLK1 proteolytic activity.

Glycoforms and Mixtures Thereof. Certain embodiments comprise a mixture of one or more KLK1 polypeptide glycoforms, including compositions (for example, formulations, solutions, dosage forms) and that comprise defined ratios of double and triple glycosylated KLK1 polypeptides (see, for example, U.S. Application No. 2015/0196624, incorporated by reference in its entirety).

Human kallikrein has three potential Asn-linked (N-linked) glycosylation sites at residues 78, 84, and 141, relative to the mature amino acid sequence shown, for example, in SEQ ID NO: 3 or 4, as well as putative O-linked glycosylation sites. However, O-linked glycosylation is not detected in naturally-occurring KLK1. By SDS-PAGE analysis, KLK1 polypeptides glycosylated at all three positions (positions 78, 84, and 141) are detected as the high molecular weight band and are referred to herein as the high-molecular weight, triple glycosylated glycoform of KLK1 (or “high glycoform” or “triple glycoform” KLK1). By SDS-PAGE analysis, KLK1 polypeptides glycosylated at only two of three available positions (positions 78 and 84) are detected as a low molecular weight band and are referred to herein as the low-molecular weight, double glycosylated glycoform of KLK1 (or as “low glycoform” or “double glycoform” KLK1).

Certain compositions therefore comprise a mixture of KLK1 glycoforms at a defined ratio, for example, comprising a first KLK1 polypeptide and a second KLK1 polypeptide, wherein the first KLK1 polypeptide has three glycans attached at the three different positions available for glycosylation in the polypeptide, and wherein the second KLK1 polypeptide has two glycans attached at only two of the three different positions available for glycosylation in the polypeptide. In certain embodiments, the first and second KLK1 polypeptides are present at a ratio of about 45:55 to about 55:45, including, for example, about 46:54, about 47:53, about 48:52, about 49:51, about 51:49, about 52:48, about 53:47, and about 54:46, including all integers and decimal points in between. In specific embodiments, the first and second KLK1 polypeptides are present at a ratio of about 50:50. In some embodiments, the ratio of the first and second KLK1 polypeptides is not about 60:40. In some embodiments, the ratio of the first and second KLK1 polypeptides is not about 40:60. In certain embodiments, the composition is free or substantially free of other glycosylated isoforms (glycoforms) of KLK1.

Some compositions comprise a triple glycoform of a KLK1 polypeptide and a double glycoform of a KLK1 polypeptide, wherein the triple glycoform and the double glycoform are present at a ratio of about 45:55 to about 55:45 including, for example, about 46:54, about 47:53, about 48:52, about 49:51, about 51:49, about 52:48, about 53:47, and about 54:46. In some embodiments, the triple glycoform and the double glycoform are present at a ratio of about 50:50. In some embodiments, the ratio of the triple glycoform and double glycoform is not about 60:40. In some embodiments, the ratio of the triple glycoform and double glycoform is not about 40:60. In certain embodiments, the composition is free or substantially free of other glycosylated isoforms (glycoforms) of KLK1.

In specific embodiments, the formulation of one or more KLK1 polypeptides is DM199. As used herein, “DM199” refers to a formulation composed of two glycoforms of a mature, human KLK1 variant polypeptide, each glycoform having the amino acid sequence set forth in SEQ ID NO: 4 (or amino acid residues 25-262 of SEQ ID NO: 2): one being a triple glycoform that has three N-linked glycans attached at residues 78, 84, and 141, and the other being a double glycoform that has two N-linked glycans attached at residues 78 and 84 but not 141 (the numbering being defined by SEQ ID NO: SEQ ID NO: 3 or 4), wherein the triple glycoform and the double glycoform are formulated at a ratio of about 50:50.

The ratios of the double and triple glycosylated isoforms of KLK1 can be detected and quantitated by a variety of methods, including high performance liquid chromatography (HPLC), which may include reversed phase (RP-HPLC), lectin affinity chromatography and lectin affinity electrophoresis. The preparation and characterization of KLK1 glycoform mixtures is described in U.S. Application No. 2015/0196624, incorporated by reference in its entirety.

Purity. In some embodiments, the “purity” of a composition (for example, formulation, solution, dosage form) is characterized, for example, by the amount (e.g., total amount, relative amount, percentage) of host cell protein(s), host cell DNA, endotoxin, and/or percentage single peak purity by SEC HPLC. In some instances, the purity of a composition is characterized by the amount (e.g., percentage) of KLK1 polypeptide relative to other components, for example, any one or more of the foregoing.

In some embodiments, the purity of a composition is characterized relative to or by the levels or amount of host cell proteins. The host cells used for recombinant expression may range from bacteria and yeast to cell lines derived from mammalian or insect species. The cells contain hundreds to thousands of host cell proteins (HCPs) and other biomolecules that could contaminate the final product. The HCP may be secreted along with the protein of interest, or released by accidental lysing of the cells, and may contaminate the protein of interest. Two types of immunological methods may be applied to HCP analysis: Western blotting (WB) and immunoassay (IA), which includes techniques such as ELISA and sandwich immunoassay or similar methods using radioactive, luminescent, or fluorescent reporting labels. Compositions of the present invention may include host cell protein of less than about 500, less than about 400, less than about 300, less than about 200, less than about 100 or less than about 50 ng/mg total protein.

In some instances, purity is characterized relative to or by the levels or amount of host cell DNA. Detection of residual host cell DNA may be performed by Polymerase Chain Reaction (PCR) with a variety of primers for sequences in the host cell genome. Residual host cell DNA is generally reported as being below a certain threshold level, but may also be quantitated with a rPCR method. Compositions of the present invention may include host cell deoxyribonucleic acid (DNA) of less than about 100, less than about 90, less than about 80, less than about 70, less than about 60, less than about 50, less than about 40, less than about 30, less than about 20, or less than about 10 pg/mg total protein.

In certain embodiments, purity is characterized relative to or by the amount or levels of endotoxin. As noted herein, endotoxin is extremely potent, heat stable, passes sterilizing membrane filters, and is present everywhere bacteria are or have been present. An Endotoxin Unit (EU) is a unit of biological activity of the USP Reference Endotoxin Standard.

The bacterial endotoxins test (BET) is a test to detect or quantify endotoxins from Gram-negative bacteria using amebocyte lysate (white blood cells) from the horseshoe crab (Limulus polyphemus or Tachypleus tridentatus). Limulus amebocyte lysate (LAL) reagent, FDA approved, is used for all USP endotoxin tests. There are at least three methods for this test: Method A, the gel-clot technique, which is based on gel formation; Method B, the turbidimetric technique, based on the development of turbidity after cleavage of an endogenous substrate; and Method C, the chromogenic technique, based on the development of color after cleavage of a synthetic peptide-chromogen complex.

At least two types of endotoxin tests are described in the USP<85>BET. Photometric tests require a spectrophotometer, endotoxin-specific software and printout capability. The simplest photometric system is a handheld unit employing a single-use LAL cartridge that contains dried, pre-calibrated reagents; there is no need for liquid reagents or standards. The FDA-approved unit is marketed under the name of Endosafe®-PTS™. The device requires about 15 minutes to analyze small amounts of sample, a 25 μL aliquot from CSP diluted in a sterile tube, and to print out results. In contrast, gel-clot methods require a dry-heat block, calibrated pipettes and thermometer, vortex mixer, freeze-dried LAL reagents, LAL Reagent Water (LRW) for hydrating reagents and depyrogenated glassware. In this clot test, diluted sample and liquid reagents require about an hour for sample and positive-control preparation and an hour's incubation in a heat block; results are recorded manually. Thus, the simplicity and speed of the automated system make it ideally suited to the pharmacy setting.

In some instances, the purity of a composition is characterized by the degree of aggregation. For instance, the degree of aggregation of KLK1 can be determined by Size-exclusion chromatography (SEC), which separates particles on the basis of size. It is a generally accepted method for determining the tertiary structure and quaternary structure of purified proteins. SEC is used primarily for the analysis of large molecules such as proteins or polymers. SEC works by trapping these smaller molecules in the pores of a particle. The larger molecules simply pass by the pores as they are too large to enter the pores. Larger molecules therefore flow through the column quicker than smaller molecules, that is, the smaller the molecule, the longer the retention time. Certain embodiments are also substantially free of aggregates (greater than about 95% appearing as a single peak by SEC HPLC). Certain embodiments are free of aggregates with greater than about 96%, about 97%, about 98%, or about 99%, appearing as a single peak by SEC HPLC.

In certain embodiments, the “purity” of the KLK1 polypeptide(s) in a composition is specifically defined. For instance, certain embodiments comprise one or more hKLK1 polypeptides that are at least about 80, at least about 85, at least about 90, at least about 91, at least about 92, at least about 93, at least about 94, at least about 95, at least about 96, at least about 97, at least about 98, at least about 99, or 100% pure, including all decimals in between, relative to other components. Purity can be measured, for example and by no means limiting, by high performance liquid chromatography (HPLC), a well-known form of column chromatography used frequently in biochemistry and analytical chemistry to separate, identify, and quantify compounds.

In certain embodiments, a composition has one or more of the following determinations of purity: less than about 1 EU endotoxin/mg protein, less that about 100 ng host cell protein/mg protein, less than about 10 pg host cell DNA/mg protein, and/or greater than about 95% single peak purity by SEC HPLC.

In some instances, a composition is formulated with pharmaceutically acceptable excipients, diluents, adjuvants, or carriers, for instance, to optimize stability and achieve isotonicity. In certain aspects, the pH is near physiological pH or about pH 7.4, including about pH 6.5, about 7.0, about 7.1, about 7.2, about 7.3, about 7.4, about 7.5, about 7.6, about 7.7, about 7.8, about 7.9, about 8.0, about 8.5, or any range thereof. In some embodiments, a composition comprises a KLK1 polypeptide in combination with a physiologically acceptable carrier. Such carriers include pharmaceutically acceptable carriers, excipients, or stabilizers which are nontoxic to the cell or mammal being exposed thereto at the dosages and concentrations employed. Methods of formulation are well known in the art and are disclosed, for example, in Remington: The Science and Practice of Pharmacy, Mack Publishing Company, Easton, Pa., Edition 21 (2005).

The phrase “physiologically-acceptable” or “pharmaceutically-acceptable” refers to molecular entities and compositions that do not produce a significant allergic or similar untoward reaction when administered to a human. Typically, such compositions are prepared as injectables, either as liquid solutions or suspensions; solid forms suitable for solution in, or suspension in, liquid prior to injection can also be prepared. The preparations can also be emulsified.

As used herein, “carrier” includes any and all solvents, dispersion media, vehicles, coatings, diluents, isotonic and absorption delaying agents, buffers, carrier solutions, suspensions, colloids, and the like. Except insofar as any conventional media or agent is incompatible with the active ingredient, its use in the compositions is contemplated.

In certain embodiments, the pharmaceutical composition is formulated and/or administered at a total KLK1 polypeptide dosage of about 0.1 to about 0.25 or 0.5 μg/kg to about 10.0 μg/kg or to about 15 μg/kg or to about 20 μg/kg, optionally about 0.1, 0.2, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10, 11, 12, 13, 14, 15, 16, 17, 18, 19 or 20 μg/kg. In specific embodiments, the pharmaceutical composition is formulated or administered at a total KLK1 polypeptide intravenous dosage of about 0.1 or 0.25 or 0.5 μg/kg (for example, as an approximately 30 minute IV infusion). In particular embodiments, the pharmaceutical composition is formulated or administered at a total KLK1 polypeptide subcutaneous dosage of about 3.0 μg/kg or up to about 15 μg/kg.

In certain instances, the administration of a composition achieves in the subject a therapeutically-effective serum level of the one or more KLK1 polypeptides. In some instances, the therapeutically-effective serum level is about or at least about 1.0 to about or at least about 5.0 ng/ml, or about or at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mg/ml, including all ranges in between. In some instances, IV administration achieves a therapeutically-effective serum level of the one or more KLK1 polypeptides in about or less than about 0.5, 1, 2, 3, 4, 5, 6, 7, 8, 9, 10, 11, 12, 13, 14, 15, 16, 17, 18, 19, or 20 hours following administration.

Certain embodiments include a dosage regimen of administering one or more compositions at defined intervals over a period of time. For example, certain dosage regimens include intravenously administering once or twice a day, once or twice every two days (e.g., once a day every other day), once or twice every three days (e.g., once a day every third day following an initial or earlier administration), once or twice every four days, once or twice every five days, once or twice every six days, once or twice every week, once or twice every other week. Certain dosage regimens include subcutaneously administering once a day every two or three or four days (e.g., once a day every third day following an initial or earlier administration). For instance, in specific embodiments, the pharmaceutical composition is administered as a dosing regimen of about a total KLK1 polypeptide intravenous dosage of about 0.1 or 0.25 or 0.5 μg/kg per day, and a total KLK1 polypeptide subcutaneous dosage of about 3.0 μg/kg or up to about 15 μg/kg every two, three or four, days, optionally every three days. In some embodiments, a dosing regimen maintains the therapeutically-effective serum level of the one or more KLK1 polypeptides, for example, over a period of one, two, three, four, five weeks, or six weeks or more, or one or two, or three, or four, five, or six more months or more.

Some variation in dosage will necessarily occur depending on the condition of the subject being treated. The person responsible for administration will, in any event, determine the appropriate dose or dosing regimen for the individual subject. Moreover, for human administration, preparations should meet sterility, pyrogenicity, and general safety and purity standards as required by the FDA. In some instances, preparation are substantially endotoxin-free or pyrogen-free, as described herein. According to the FDA Guidance for Industry; Estimating the Maximum Safe Starting Dose in Initial Clinical Trial for Therapeutics in Adult Healthy Volunteers (July 2005), Appendix D: Converting animal doses to human equivalent doses. A human equivalent dose is 1/7 the rat dose and a human equivalent dose is 1/12 a mouse dose.

In some embodiments, administering a composition improves one or more clinical parameters in the patient. In certain embodiments, the pregnancy disorder is FGR, and the one or more clinical parameters are selected from achieving serum KLK1 levels of about 1-5 ng/ml in the pregnant subject and increased fetal body weight for gestational age (optionally increased EFW percentile of about or greater than 10^{th}, 15^{th}, 20^{th}, 25^{th}, 30^{th}, 35^{th}, 40^{th}, 50^{th}, or greater percentile for gestational age). In some embodiments, the pregnancy disorder is preeclampsia, and the one or more clinical parameters are selected from achieving serum KLK1 levels of about 1-5 ng/ml, decreased systolic and/or diastolic blood pressure, and decreased proteinuria in the pregnant subject. Any one or more of the foregoing clinical parameters can be measured according to routine clinical techniques in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Maternal and Fetal Safety

Pre-clinical Reprotox® studies were performed to assess the safety of DM199 in fetuses. DM199 was administered by subcutaneous injection to time-mated Crl:CD (SD) Sprague Dawley female rats once daily beginning on GD 7 and continuing through GD 17 at dose levels of 1.44, 2.88, or 3.69 mg/kg/day.

Mortality. All rats survived to scheduled euthanasia on GD 17/18 or GD 21 in the Toxicokinetic Phase and Main Study dose groups, respectively.

Clinical Observations. There were no test article-related clinical findings observed in rats at any dose level. Beginning of GD 8 and persisting as late as GD 21, skin scabbing was observed in 7, 4, 6, and 3 in the 0 (Control), 1.44, 2.88, and 3.69 mg/kg/day DM199-treated groups. This finding did not occur in a dose-related manner and was ascribed to the route of administration (subcutaneous injection). The only other clinical findings observed were thin fur cover and skin abrasion. These findings were considered unrelated to DM199 because: 1) the number of rats affected was similar to controls and/or; 2) the observations were limited to one or two rats in any dose group.

Maternal Body Weight and Body Weight Gains. There were no DM199-related effects on mean maternal body weight, mean maternal body weight gain, mean maternal adjusted body weight, or mean maternal adjusted body weight gain during the study. All main study animals gained weight for each weighing interval throughout gestation. On GD 21, mean maternal body weight in the 1.44, 2.88, and 3.69 mg/kg/day groups was 99.0%, 101%, and 101% of the control group mean, respectively.

Mean adjusted maternal body weight gain in the 1.44, 2.88, and 3.69 mg/kg/day groups was 90.4%, 97.7%, 88.6% of the control group mean, respectively. These differences were considered unrelated to treatment as they did not occur in a dose-related manner, and mean maternal adjusted body weight and mean maternal body weight gains in the DM199-treated groups were comparable to corresponding control group values.

Maternal Food Consumption. There were no DM199-related effects on mean maternal food consumption throughout the study. Mean maternal food consumption in the 1.44, 2.88, and 3.69 mg/kg/day groups during the interval of GD 7 to GD 18 was 98.6%, 103%, and 102% of the mean maternal control group consumption, respectively, and 98.7%, 103%, and 102% of control consumption over the entire study period (GD 7 to GD 21), respectively.

Maternal Necropsy Observations. There were no macroscopic findings observed at the scheduled necropsy.

Ovarian and Uterine Examination. Pregnancy was confirmed in 22, 22, 22, and 22 females in the 0 (Control), 1.44, 2.88, and 3.69 mg/kg/day DM199-treated groups, respectively, and each of these rats was examined for ovarian and uterine contents on GD 21. Mean fetal body weights (combined, male, and female) in the 1.44, 2.88, and 3.69 mg/kg/day DM199-treated groups were comparable to those of the control group, ranging from 102% to 103% of their respective control group values.

There were no DM199-related effects on any ovarian, uterine, or litter parameters at any dose level. The mean number of corpora lutea, implantation sites, percent pre-implantation loss, percent post-implantation loss, live or dead fetuses, early and late resorptions, and fetal sex ratio were similar among the four dose groups. Any differences were slight, did not occur in a dose-related manner, and were not attributed to treatment with DM199.

Fetal Examinations. Fetal observations were defined as: 1) malformations (irreversible changes that occur at low incidences in this species and strain); 2) variations (common findings in this species and strain and reversible delays or accelerations in development) or incidental (minor changes in coloration, mechanical damage to specimen, etc.). Litter means were calculated for specific fetal ossification sites as part of the evaluation of the degree of fetal ossification.

Fetal evaluations were based on 248, 247, 251, and 256 live GD 21 Caesarean-delivered fetuses in 22, 22, 22, and 22 litters in the 0, 1.44, 2.88, and 3.69 mg/kg/day dose groups, respectively. Each of these fetuses was examined for external abnormalities. Of these respective fetuses, 117, 117, 119, and 122 fetuses were examined for visceral abnormalities, and 131, 130, 132, and 134 fetuses were examined for skeletal abnormalities and fetal ossification site averages.

There were no DM199-related malformations or variations at the fetal external, visceral, and skeletal examinations at any dose level. All fetal abnormalities observed did not occur in a dose-related manner, were within historical control ranges for similarly powered studies in this species and strain, and were considered to be spontaneous, background findings.

External Examinations. There were no DM199-related malformations or variations detected at fetal external examination. All external abnormalities that were observed were considered unrelated to DM199 because: 1) the finding was limited to the control group; and/or 2) the finding was not dose-dependent.

Visceral Examinations. There were no DM199-related malformations or variations detected at fetal visceral examination. All visceral abnormalities that were observed were considered unrelated to DM199 because: 1) the finding was limited to the control group; and/or 2) the finding was not dose-dependent

Skeletal Examinations. There were no DM199-related malformations or variations detected at fetal skeletal examination. All visceral abnormalities that were observed were considered unrelated to DM199 because: 1) the finding was limited to the control group; and/or 2) the finding was not dose-dependent.

The mean number of fetal ossification sites was similar across all groups with the exception of the mean number of tarsal ossification sites. The mean numbers of tarsal ossification sites for the 0, 1.44, 2.88, and 3.69 mg/kg/day dose group fetuses were 0.06, 0.01, 0.02, and 0.01, respectively. Because all of the mean values were within historical control limits for similarly powered studies in this species and strain and there were no other remarkable differences from control group ossification sites at any dose level, these differences were considered spontaneous and unrelated to treatment with DM199.

Concentration Observations. Plasma concentrations of DM199 were below the LLOQ (<50.0 ng/ml) for all samples from the control group collected at 4 hr post-dose on GDs 7 and 17, and for all GD 7 pre-dose samples collected from the DM199-treated animals.

Following once daily subcutaneous administration, DM199 mean plasma concentrations were generally quantifiable throughout the 24 hours sampling, on both GDs 7 and 17, including at pre-dose on GD 17. The exceptions were for Group 2 (1.44 mg/kg) for which mean plasma concentrations were below the LLOQ at 0.25 hr post-dose and were quantifiable from 1 to 24 hr post-dose on GD 7 and were below the LLOQ at pre-dose on GD 17. Of note, high inter-animal variability was observed for all dose groups on GD 17 with % CV ranging from 33.5% to 173%.

Toxicokinetic Evaluations. Across all dose levels, the tₘₐₓ was observed at 12 hours post-dose on GD 7 and at 1 or 4 hours post-dose on GD 17. Due to insufficient time points following Cmax or reporting criteria not met, the terminal elimination phase could not be determined for any profiles.

Even with the small increase in doses, exposures (Cₘₐₓ and AUCₜₗₐₛₜ) of DM199 increased with dose levels on both sampling occasions except between 2.88 and 3.69 mg/kg on GD 7 for which exposure remained approximately similar. The increase in exposure on GD 7 was close to dose proportional between 1.44 and 2.88 mg/kg for Cₘₐₓ and AUCₜₗₐₛₜ. On GD 17, the increase in exposure was approximately dose proportional between 1.44 and 3.69 mg/kg for Cₘₐₓ Whereas for AUCₜₗₐₛₜ, the increase in exposure was greater than dose proportional between 1.44 and 2.88 mg/kg and dose proportional between 2.88 and 3.69 mg/kg.

Following repeated dosing, the Cₘₐₓ and AUCₜₗₐₛₜ values were lower on GD 17 compared to GD 7. The accumulation ratios (RAUC) for AUCₜₗₐₛₜ increased with doses and were 0.250, 0.436 and 0.608 at 1.44, 2.88 and 3.69 mg/kg, respectively.

Conclusions. There were no negative maternal effects related to administration of DM199. There were no negative effects on embryo-fetal viability or mean fetal body weights in the 1.44, 2.88, or 3.69 mg/kg/day dose groups. DM199 did not produce any fetal external, visceral, or skeletal malformations or variations. Based on these results, the non-observed-adverse-effect level (NOAEL) for maternal and developmental toxicity was considered to be 3.69 mg/kg/day under the conditions of this study (corresponds to a maternal Cₘₐₓ of 546 ng/ml and a maternal AUCₜₗₐₛₜ of 9280 hr*ng/ml on GD 17).

### Example 2

Effects of DM199 on Reducing Blood Pressure, Proteinuria, and Blood Glucose Separate human clinical studies were performed to assess the efficacy of DM199 in reducing blood pressure, proteinuria, and elevated blood glucose, each of which can be a hallmark of preeclampsia. In one study, a cohort of hypertensive patients with baseline urine albumin-creatine ratio (UACR) of ≥500 g/kg was treated with 2 μg/kg or 5 μg/kg of DM199. The results on systolic blood pressure (mmHg) are summarized in Table E1 below, and the results on diastolic blood pressure (mmHg) are shown in Table E2 below.

TABLE E1
Systolic Blood Pressure Summary - Cohort 1

| | Observed/ | | DM-199 | | DM-199 | | | |
|---|---|---|---|---|---|---|---|---|
| | Change from | | 2 ug/kg | | 5 ug/kg | | Overall | |
| Visit | Baseline | Statistics | (N = 10) | | (N = 10) | | (N = 20) | |
| Week 14 | Observed | n | 9 | | 7 | | 16 | |
| Day 95 | | Mean (SD) | 136.6 | (10.38) | 137.7 | (10.73) | 137.1 | (10.19) |
| | | Median | 136.0 | | 135.0 | | 135.5 | |
| | | Min, Max | 119, 157 | | 125, 158 | | 119, 158 | |
| | Change from | n | 9 | | 7 | | 16 | |
| | Baseline | Mean (SD) | −16.6 | (34.26) | −2.4 | (17.23) | −10.4 | (28.24) |

| Median | −5.0 | | −2.0 | | −4.0 |
|---|---|---|---|---|---|
| Min, Max | −72, 26 | | −37, 16 | | −72, 26 |
| LS Means | −5.00 | (5.107) | −1.91 | (5.504) | |

(SEM)

The LS means were estimated using a mixed model with baseline, cohort and dose group as fixed effects and visit as random effect.
SEM = Standard error of the mean

TABLE E2
Diastolic Blood Pressure Summary - Cohort 1

| | Observed/ | | DM-199 | | DM-199 | | | |
|---|---|---|---|---|---|---|---|---|
| | Change from | | 2 ug/kg | | 5 ug/kg | | Overall | |
| Visit | Baseline | Statistics | (N = 10) | | (N = 10) | | (N = 20) | |
| Week 14 | Observed | n | 9 | | 7 | | 16 | |
| Day 95 | | Mean (SD) | 84.3 | (7.02) | 81.4 | (12.43) | 83.1 | (9.50) |
| | | Median | 85.0 | | 86.0 | | 85.5 | |
| | | Min, Max | 72, 96 | | 60, 95 | | 60, 96 | |
| | Change from | n | 9 | | 7 | | 16 | |
| | Baseline | Mean (SD) | −10.6 | (21.17) | −1.9 | (10.27) | −6.8 | (17.35) |

| Median | 1.0 | | −4.0 | | −3.0 |
|---|---|---|---|---|---|
| Min, Max | −49, 8 | | −13, 19 | | −49, 19 |
| LS Means | −2.05 | (3.277) | 0.06 | (3.478) | |

(SEM)

The LS means were estimated using a mixed model with baseline, cohort and dose group as fixed effects and visit as random effect.
SEM = Standard error of the mean

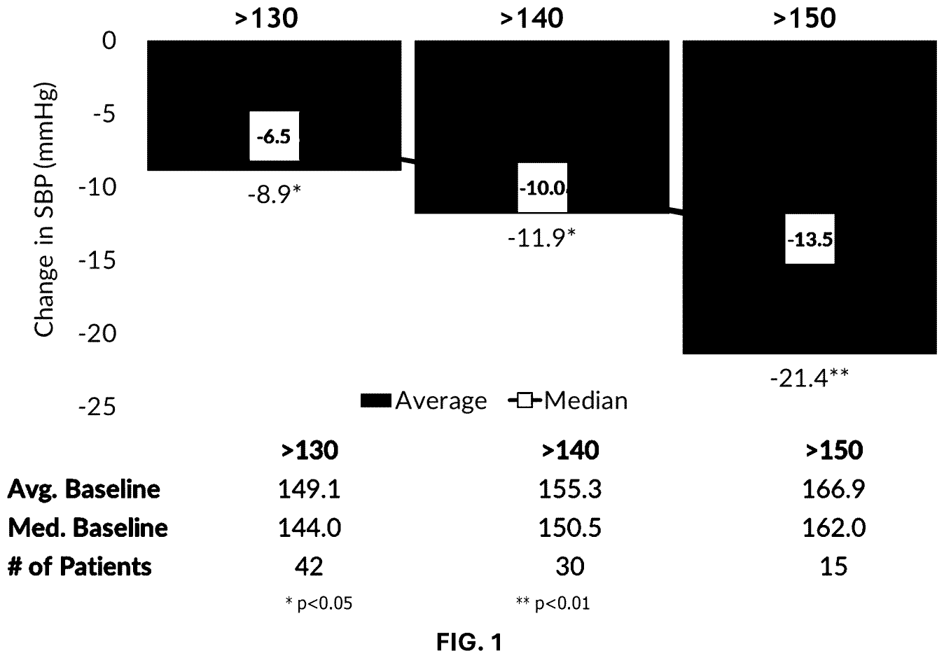

Overall, there was clinically relevant and meaningful 10.4 point decrease in systolic blood pressure combined with a 6.8 point decrease in diastolic blood pressure. FIG. 1 also summarizes DM199-mediated reductions in systolic blood pressure in cohorts of hypertensive patients at day 95 of treatment.

The results on proteinuria are summarized in Table E3 below. Here, a responder was defined as a participant who has at least 30% reduction in UACR (over the course of the study) and no worsening in eGFR (defined as a shift of at least 1 category down from baseline over the course of the study).

TABLE E3
Composite Responder Analysis by Baseline UACR Category - Cohort 1

| | | DM-199 | | DM-199 | | | |
|---|---|---|---|---|---|---|---|
| | | 2 ug/kg | | 5 ug/kg | | Overall | |
| Visit | | (N = 10) | | (N = 10) | | (N = 20) | |
| Week 6 | Responders, n (%) | 3 | (60.0) | 1 | (25.0) | 4 | (44.4) |
| | Participants at | 5 | | 4 | | 9 | |
| | Visit | | | | | | |
| Week 8 | Responders, n (%) | 3 | (100.0) | 1 | (25.0) | 4 | (57.1) |
| | Participants at | 3 | | 4 | | 7 | |
| | Visit | | | | | | |
| Week 10 | Responders, n (%) | 3 | (75.0) | 1 | (25.0) | 4 | (50.0) |
| | Participants at | 4 | | 4 | | 8 | |
| | Visit | | | | | | |
| Week 12 | Responders, n (%) | 3 | (75.0) | 1 | (25.0) | 4 | (50.0) |
| | Participants at | 4 | | 4 | | 8 | |
| | Visit | | | | | | |
| Week 14 | Responders, n (%) | 3 | (75.0) | 1 | (33.3) | 4 | (57.1) |
| | Participants at | 4 | | 3 | | 7 | |
| | Visit | | | | | | |
| Week 16 | Responders, n (%) | 3 | (100.0) | 1 | (33.3) | 4 | (66.7) |
| | Participants at | 3 | | 3 | | 6 | |
| | Visit | | | | | | |
| End of | Responders, n (%) | 4 | (80.0) | 1 | (20.0) | 5 | (50.0) |

| Study [1] | Participants at | 5 | 5 | 10 |
|---|---|---|---|---|
| | Visit | | | |

UACR = urine albumin creatinine ratio; eGFR = estimated glomerular filtration rate, calculated using Chronic Kidney Disease Epidemiology Collaboration formula.
Percentages use the total number of participants with non-missing eGFR and UACR for each visit as the denominator.
[1] End of Study is the last available post-baseline visit for all subjects under the current study population where both eGFR and UACR are non-missing.

The results in Table E3 show a reduction in proteinuria in the Composite Responder analysis for cohort 1 (hypertensive African Americans), particularly at the 2 ug dose, in patients with baseline proteinuria of 500 g/kg or higher. The 30% threshold is based on the two sister studies supporting 30% UACR reduction as a surrogate endpoint for FDA conditional approval. Overall 50% of patients were responders.

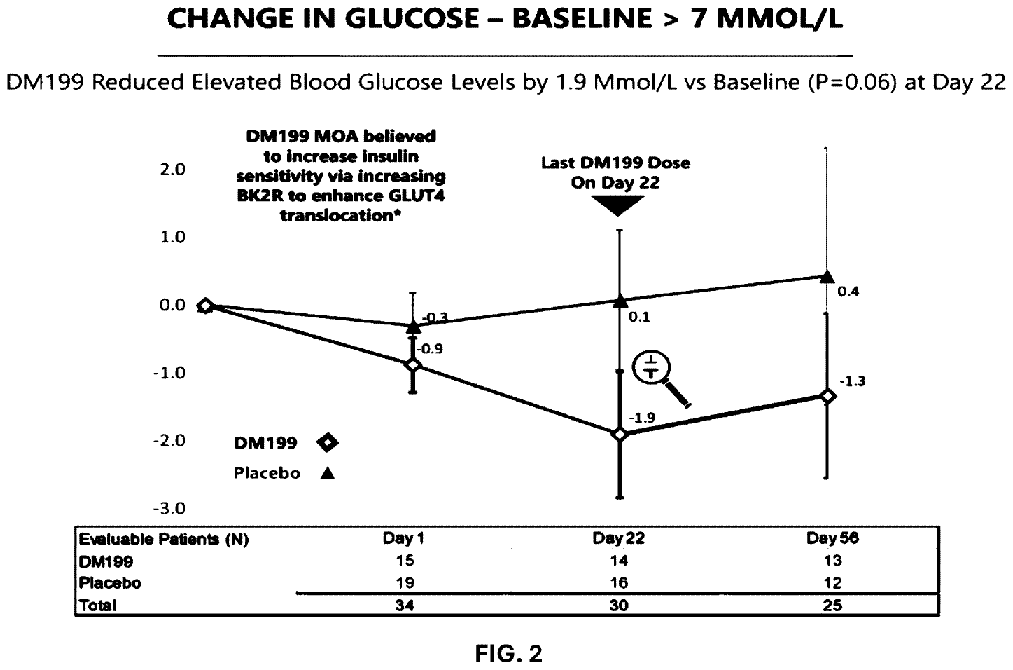

In another clinical study, DM199 was administered at 1 μg/kg as a single intravenous infusion, followed by a 3 μg/kg subcutaneous (SC) dose 2-12 hours after the intravenous infusion, and then 3 μg/kg SC doses every 72 hours for the remainder of the 22 day treatment period. FIG. 2 shows that DM199 reduces blood glucose levels in human patients.

### Example 3

### Placental Transfer Study

A placental transfer study was performed to asses the potential of KLK1 (DM199) to cross the placental barrier. Pregnant rats were administered large doses of DM199 and sacrificed at different timepoints after administration. DM199 levels were subsequently measured in the serum of both the dams (maternal, n=30) and the pups (fetal, n=24).

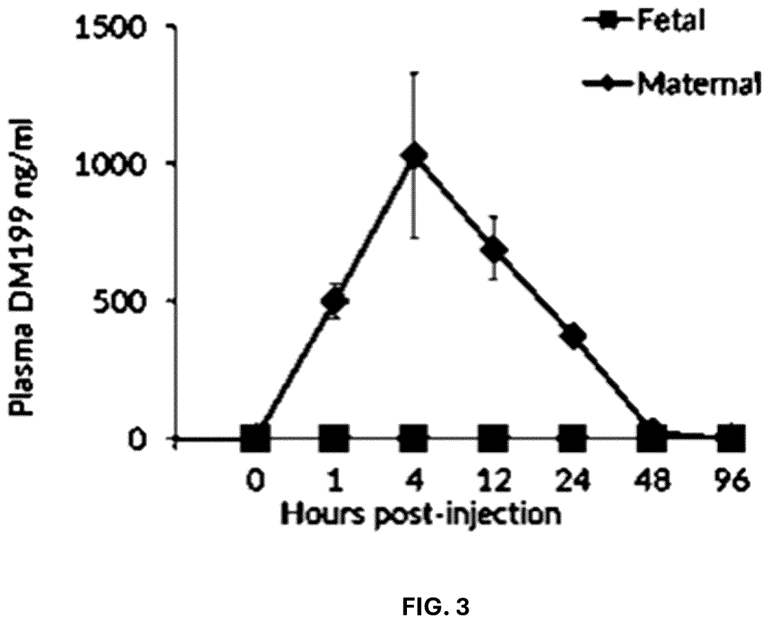

The results are provided in FIG. 3, which show a clear pharmacokinetic curve in the dams (up to about 1000 ng/ml) but no detectable levels of DM199 in the pups at any timepoint. Values below the level of quantification were reported as zero. This result provides further evidence of the potential safety of DM199 in treating complications of pregnancy because it does not cross the placental barrier.

### Example 4

### Cohort of Women with Cohort of Women with Preeclampsia, Severity Hypertensive and for Planned Birth

Phase 2a clinical trials are being performed on a cohort that will help exclude the possible risk of DM199 causing uterine contractions, as per Table E4 below.

TABLE E4
DM199 Phase 2a - Cohort of women with preeclampsia,
severity hypertensive and for planned birth

| | |
|---|---|
| P | 20 women with preeclampsia with severe hypertension, clinical decision made |
| | for planned birth within 72 hrs |
| I | IV dosing until birth |
| C | No controls. This is a single arm trial |
| O | Safety and tolerability |
| | Primary safety outcomes |
| | Confirm absence of uterine contractions |
| | Confirm absence of acute hypotension |
| | Confirm very low/undetectable levels of DM199 in umbilical cord blood |
| | Efficacy |
| | Primary efficacy outcome: |
| | Change in BP from baseline/reduced need for BP meds |
| | Exploratory: |
| | Changes in Doppler blood flows (uterine artery Doppler (maternal) and |
| | umbilical cord, middle cerebral artery (fetal)) |
| | Changes in routine biochemical indices (Hemoglobin, liver function |
| | tests, renal function tests urea, creatinine; changes in proteinuria) |
| | Changes in biomarkers of possible disease severity (sFlt/PIGF, +/−NO) |
| | Other clinical neonatal and maternal adverse outcomes that are |
| | associated with preeclampsia |

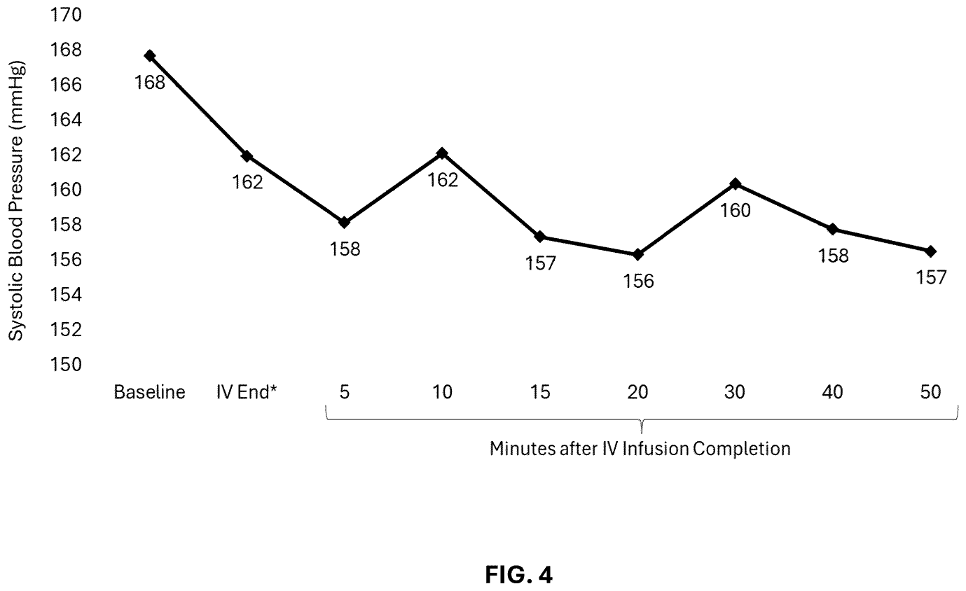

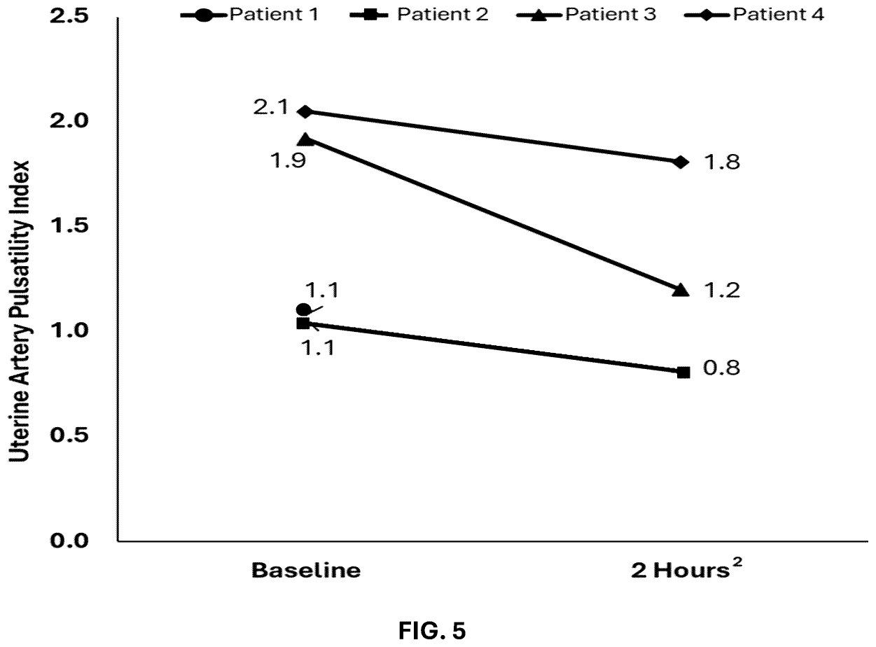

FIG. 4 shows the initial clinical results on reducing blood pressure. Here, a 30 minute intravenous infusion (0.1 mg/kg) was shown to reduce the average systolic blood pressure in 5 (five) patients that have been dosed to date. FIG. 5 and Table E5 below show that administration of DM199 reduces the uterine artery pulsality index in 3 (three) patients for which this data is available, a result that evidences improved blood flow to the fetus.

TABLE E5
Uterine Artery Pulsality Index

| | | | |
|---|---|---|---|
| Patient | Baseline | 2 Hours | % Change |

| | | |
|---|---|---|
| 1 | 1.1 | N/A - Emergency C-Section |

| 2 | 1.1 | 0.8 | −22% |
|---|---|---|---|
| 3 | 1.9 | 1.2 | −38% |
| 4 | 2.1 | 1.8 | −12% |
| 5 | Not Available | | |
| Average* | 1.7 | 1.3 | −24% |

*Excludes patient 1

Also, there have been no adverse events or serious adverse events for the DM199 pregnancy complications study. The trial drug has been well tolerated by all participants.

Given that birth within 72 hours is already planned, the adverse impact of provoking spontaneous preterm birth is removed. This trial will and has provided early data on 1) the positive effects on blood pressure and/or 2) levels of DM199 in the fetal circulation at birth. It will and has also shown improvements in one or more clinical parameters, for instance, by achieving serum KLK1 levels of about 1-5 ng/ml, decreased systolic/diastolic blood pressure, and/or decreased uterine artery pulsality index.

### Example 5

### Cohort of Women with Preterm Preeclampsia, for Expectant Management

Phase 2a clinical trials are being performed on a cohort that will obtain further data on the safety of repeated dosing, and provide further reassurance DM199 will not cause uterine contractions, as per Table E6 below.

TABLE E6
DM199 Phase 2a - Cohort of women with preterm
preeclampsia, for expectant management

| | |
|---|---|
| P | 20 women with preeclampsia diagnosed between 26-32 weeks gestation, for |
| | expectant management |
| I | Serial IV dosing until birth (or sc) |
| C | No controls. This is a single arm trial |
| O | Safety and tolerability |
| | Primary safety outcomes |
| | Confirm absence of uterine contractions with serial dosing |
| | Confirm absence of acute hypotension |
| | Confirm very low/undetectable levels of DM199 in umbilical cord blood |
| | with serial dosing |
| | Efficacy |
| | Primary efficacy outcome: |
| | Change in BP from baseline/reduced need for BP meds |
| | Exploratory: |
| | Changes in Doppler blood flows (uterine artery Doppler (maternal) and |
| | umbilical cord, middle cerebral artery (fetal)) |
| | Changes in routine biochemical indices (Hemoglobin, liver function |
| | tests, renal function tests urea, creatinine; changes in proteinuria) |
| | Changes in biomarkers of possible disease severity (sFlt/PIGF, +/−NO) |
| | Other clinical neonatal and maternal adverse outcomes that are |
| | associated with preeclampsia |
| | Changes in flow mediated dilatation (non-invasive instrument used to |
| | assess maternal vessel dysfunction) |

This trial will also provide further early data on 1) the positive effects on blood pressure and 2) DM199 levels in the fetal circulation at birth. It may also generate data on 3) uterine artery Doppler changes. It will also show improvements in one or more clinical parameters, for instance, by achieving serum KLK1 levels of about 1-5 ng/ml, decreased systolic and/or diastolic blood pressure, decreased proteinuria, and/or decreased edema. Further, around 30% are expected to be <3^{rd} centile and 48% are expected to be <10^{th} centile. Hence, there should be enough participants with growth restriction to obtain positive data on placental and fetal blood flow indices.

### Example 6

### Cohort of Women with Fetal Growth Restriction

Phase 2a clinical trials are being performed on a cohort that will examine the potential of DM199 to treat women with fetal growth restriction, as per Table E7 below.

TABLE E7
Cohort of women with fetal growth restriction

| | |
|---|---|
| P | 20 women with fetal growth restriction (defined via Delphi consensus) diagnosed |
| | between 26-32 weeks gestation, for expectant management |
| | Cases that were considered viable (i.e., >750 grams) at diagnosis. |
| I | Serial IV dosing until birth (or sc) |
| C | No controls. This is a single arm trial |
| O | Safety and tolerability |
| | Primary safety outcomes |
| | Confirm absence of uterine contractions with serial dosing |
| | Confirm absence of acute hypotension |
| | Confirm very low/undetectable levels of DM199 in umbilical cord blood |
| | with serial dosing |
| | Efficacy |
| | Primary efficacy outcome: |
| | Changes in Doppler blood flows (uterine artery Doppler (maternal) and |
| | umbilical cord, middle cerebral artery (fetal)) |
| | Exploratory: |
| | Changes in fetal growth centiles |
| | Changes in biomarkers of possible disease severity (SPINT1, PIGF) |

This trial will show improvements in one or more clinical parameters of FGR, for example, by achieving serum KLK1 levels of about 1-5 ng/ml in the pregnant subject, increased fetal body weight for gestational age (including increased EFW percentile of about or greater than 10^{th}, 15^{th}, 20^{th}, 25^{th}, 30^{th}, 35^{th}, 40^{th}, 50^{th}, or greater percentile for gestational age), and/or improvements in measures of hypertension, proteinuria, and blood glucose.

1. A method of treating or preventing a pregnancy disorder in a pregnant subject, comprising administering to the subject a pharmaceutical composition comprising one or more tissue kallikrein 1 (KLK1) polypeptides.

2. The method of claim 1, wherein the pregnancy disorder is fetal growth restriction (FGR) of the fetus in the pregnant subject.

3-14. (canceled)

15. The method of claim 1, wherein the pregnancy disorder is preeclampsia, eclampsia, or post-partum preeclampsia.

16. The method of claim 15, wherein the subject has new-onset hypertension.

17. (canceled)

18. The method of claim 15, wherein
the subject has proteinuria.

19. (canceled)

20. The method of claim 15, wherein the subject has edema (optionally pulmonary edema or ankle edema, optionally pitting type).

21. The method of claim 15, wherein the subject has a condition or history of kidney dysfunction (optionally oliguria, elevated creatinine levels), impaired liver function, thrombocytopenia (optionally a platelet count <about 100,000/μl), and/or cerebral or visual disturbances.

22. The method of claim 15, wherein the preeclampsia is early-onset (<about 32 weeks of pregnancy), optionally at or after about 20 weeks of pregnancy.

23. The method of claim 15, wherein the preeclampsia is late-onset (>about 32 weeks of pregnancy).

24. The method of claim 15, wherein the subject has non-severe preeclampsia.

25. The method of claim 15, wherein the subject has severe preeclampsia.

26. The method of claim 25, wherein the severe preeclampsia is HELLP syndrome.

27. The method of claim 15, wherein the subject with preeclampsia, eclampsia, or post-partum preeclampsia has increased serum levels of soluble fms-like tyrosine kinase 1 (sFlt-1), decreased serum levels of free placental growth factor (PlGF), increased ratio of sFlt-1/PlGf in serum, or any combination thereof.

28. The method of claim 27, comprising:
(a) determining serum levels of sFlt-1 and/or free PlGF in the subject; and
(b) administering the pharmaceutical composition to the subject if the subject has increased serum levels of soluble fms-like tyrosine kinase 1 (sFlt-1), decreased serum levels of free placental growth factor (PlGF), increased ratio of sFlt-1/P1Gf in serum, or any combination thereof.

29. The method of claim 1, wherein the pregnancy disorder is chronic hypertension, wherein the subject was hypertensive prior to pregnancy and was optionally undergoing hypertensive therapy that is discontinued during pregnancy.

30. The method of claim 29, wherein the hypertensive therapy is selected from angiotensin-converting-enzyme (ACE) inhibitor therapy, angiotensin II receptor blocker (ARB) therapy, and diuretic therapy optionally thiazide therapy.

31. The method of claim 15, wherein the pregnancy disorder is chronic hypertension superimposed with preeclampsia.

32. The method of claim 1, wherein the pregnancy disorder is gestational hypertension.

33. (canceled)

34. The method of claim 1, wherein the pregnancy disorder is post-partum hypertension, optionally wherein the subject has a systolic blood pressure of 140 mm Hg or more and a diastolic blood pressure of 90 mm Hg or more that begins or is present after childbirth, for example, which begins or is present within about 48 hours after childbirth, or within about 1, 2, 3, 4, 5, or 6 weeks after childbirth.

35. The method of claim 1, wherein the subject has hypertension or a history of hypertension, and wherein the method comprises administering the pharmaceutical composition as blood pressure control for a planned delivery about 72 hours before the delivery.

36. The method of claim 1, wherein the pregnancy disorder is pregnancy-associated stroke (optionally ischemic stroke or hemorrhagic stroke).

37. The method of claim 36, wherein the pregnancy-associated stroke is post-partum stroke, optionally wherein the stroke occurs within about 48 hours of childbirth, within about 1, 2, 3, 4, 5, 6, 7, 8, 9, or 10 days after childbirth, or within about 1, 2, 3, 4, 5, or 6 weeks after childbirth.

38. (canceled)

39. The method of claim 1, wherein the pharmaceutical composition comprises DM199.

40. The method of claim 1, wherein the pharmaceutical composition comprises a first KLK1 polypeptide and a second KLK1 polypeptide, wherein the first KLK1 polypeptide has three N-linked glycans attached at residues 78, 84, or 141 of KLK1 as defined by SEQ ID NO: 3 or 4 and the second KLK1 polypeptide has two N-linked glycans attached at residues 78 and 84 but not 141, and wherein the first KLK1 polypeptide and the second KLK1 polypeptides are present in the pharmaceutical composition at a ratio of about 45:55 to about 55:45.

41-44. (canceled)

45. The method of claim 40, wherein the first KLK1 polypeptide and the second KLK1 polypeptides are present in the pharmaceutical composition at a ratio of about 50:50.

46-47. (canceled)

48. The method of claim 1, wherein the hKLK1 polypeptide(s) comprise, consist, or consist essentially of SEQ ID NO: 3 or 4, or an active variant having at least about 90, 95, 96, 97, 98, or 9995% sequence identity to SEQ ID NO: 3 or 4.

49-61. (canceled)


SEQUENCE LISTING (4 sequences)

SEQ ID NO: 1 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS

SEQ ID NO: 3 - protein, 238 aa, Homo sapiens
IVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 4 - protein, 238 aa, Homo sapiens
IVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS